(19) United States
(12) Patent Application Publication
(10) Pub. No.: US 2005/0233967 A1
(43) Pub. Date: Oct. 20, 2005

(54) Compositions, systems, and methods for focusing a cell-mediated immune response

(76) Inventors: Vladamir A. Dadali, Saint-Petersburg (RU); Nina V. Karbisheva, Saint-Petersburg (RU); Emma A. Oganova, Draper, UT (US); Calvin W. McCausland, Springville, UT (US); William J. Hennen, Eagle Mountain, UT (US)

Correspondence Address: TRASK BRITT, P.O. BOX 2550, SALT LAKE CITY, UT, 84110, US

(21) Appl. No.: 11/122,430

(22) Filed: May 4, 2005

Related U.S. Application Data
(63) Continuation of application No. PCT/US03/35161, filed on Nov. 4, 2003.
(60) Provisional application No. 60/423,965, filed on Nov. 4, 2002.

(51) Int. Cl. A61K 38/18

(52) U.S. Cl. 514/12

(57) ABSTRACT

Compositions, systems, and methods for enhancing the ability of a subject to heal itself following an infection include administering a composition that includes transfer factor to a subject. Administration of such a composition or combination of compositions to a subject may result in improving the subject's overall antioxidant profile, increasing the concentration of chemical antioxidants present in the subject, increasing the efficiency with which the treated subject's enzymatic antioxidants work, increasing the efficiency and/or activity of the treated subject's detoxification enzymes, and improving cellular and molecular health of the subject.

### CROSS-REFERENCE TO RELATED APPLICATIONS

This application is a continuation of International Patent Application No. PCT/US03/35161, filed Nov. 4, 2003, designating the United States of America and published, in English, as PCT International Patent Application No. WO 2004/141071 A2 on May 21, 2004, which claims priority to U.S. Provisional Application Ser. No. 60/423,965, filed Nov. 4, 2002, the disclosures of both of which are hereby incorporated herein, in their entireties, by this reference.

### BACKGROUND OF THE INVENTION

The present invention relates generally to methods for enhancing or sustaining the ability of a subject to heal itself following an infection and, more specifically, to the use of transfer factor to enhance the ability of a subject to heal itself. More specifically, the present invention relates to systems that include at least one biologically active agent and a composition that includes transfer factor.

### Conventional Techniques for Treating Infection

Conventionally, infections have been treated by use of antibiotics, which affect cells that are exposed thereto in such a way as to kill the exposed cells. In addition to adversely affecting bacterial cells, antibiotics may also induce toxicity and kill beneficial bacteria, as well as damage or kill the cells of a treated subject.

Antibiotics have been used to treat a wide array of infections. There is a movement, however, to curb or limit their use. This is because, as medical professionals have long been aware, many bacteria evolve in such a way as to develop strains which are resistant to antibiotics. As evidence of the severity of the problem of antibiotic-resistant bacterial strains, great efforts have recently been taken to make the general public aware that antibiotics should be used judiciously.

The usefulness of antibiotics is also largely limited to bacteria, fungi, and some parasites. Very few substances are considered effective antiviral compounds. Nonetheless, many undesirable pathogenic infections and the diseases that result therefrom are caused by viruses.

For serious bacterial infections, high doses of antibiotics may be administered to an infected subject. Sometimes, bacterial infections become so severe or unresponsive or inaccessible that surgery is needed to excise the infected areas of a subject's body and, thus, to physically remove the infecting pathogen.

The use of surgery is somewhat undesirable because of the trauma and increase in oxidative stress caused thereby. As such, surgery is often used as a last resort for eliminating infections.

Although surgery, the administration of antibiotics, or both of these techniques are useful for removing infections and, thus, for permitting the body of a treated individual to heal itself, neither of these techniques is useful for enhancing the ability of a subject to heal itself.

### The Immune System and Transfer Factor

The immune systems of vertebrates are equipped to recognize and defend the body from invading pathogenic organisms, such as parasites, bacteria, fungi, and viruses. Vertebrate immune systems typically include a cellular component and a noncellular component.

The cellular component of an immune system includes the so-called “lymphocytes,” or white blood cells, of which there are several types. It is the cellular component of a mature immune system that typically mounts a primary, nonspecific response to invading pathogens, as well as being involved in a secondary, specific response to pathogens.

In the primary, or initial, response to an infection by a pathogen, white blood cells that are known as phagocytes locate and attack the invading pathogens. Typically, a phagocyte will internalize, or “eat” a pathogen, then digest the pathogen. In addition, white blood cells produce and excrete chemicals in response to pathogenic infections that are intended to attack the pathogens or assist in directing the attack on pathogens.

Only if an infection by invading pathogens continues to elude the primary immune response is a specific, secondary immune response to the pathogen needed. As this secondary immune response is typically delayed, it is also known as “delayed-type hypersensitivity.” A mammal, on its own, will typically not elicit a secondary immune response to a pathogen until about seven (7) to about fourteen (14) days after becoming infected with the pathogen. The secondary immune response is also referred to as an acquired immunity to specific pathogens. Pathogens have one or more characteristic proteins, which are referred to as “antigens.” In a secondary immune response, white blood cells known as B lymphocytes, or “B-cells,” and T lymphocytes, or “T-cells,” “learn” to recognize one or more of the antigens of a pathogen. The B-cells and T-cells work together to generate proteins called “antibodies,” which are specific for one or more certain antigens on a pathogen.

The T-cells are primarily responsible for the secondary, or delayed-type hypersensitivity, immune response to a pathogen or antigenic agent. There are three types of T-cells: T-helper cells, T-suppressor cells, and antigen-specific T-cells, which are also referred to as cytotoxic (meaning “cell-killing”) T-lymphocytes (“CTLs”), or T-killer cells. The T-helper and T-suppressor cells, while not specific for certain antigens, perform conditioning functions (e.g., the inflammation that typically accompanies an infection) that assist in the removal of pathogens or antigenic agents from an infected host.

Antibodies, which make up only a part of the noncellular component of an immune system, recognize specific antigens and, thus, are said to be “antigen-specific.” The generated antibodies then basically assist the white blood cells in locating and eliminating the pathogen from the body. Typically, once a white blood cell has generated an antibody against a pathogen, the white blood cell and all of its progenitors continue to produce the antibody. After an infection is eliminated, a small number of T-cells and B-cells that correspond to the recognized antigens are retained in a “resting” state. When the corresponding pathogenic or antigenic agents again infect the host, the “resting” T-cells and B-cells activate and, within about forty-eight (48) hours, induce a rapid immune response. By responding in this manner, the immune system mounts a secondary immune response to a pathogen; the immune system is said to have a “memory” for that pathogen.

Mammalian immune systems are also known to produce smaller proteins, known as “transfer factors,” as part of a secondary immune response to infecting pathogens. Transfer factors are another noncellular part of a mammalian immune system. Antigen-specific transfer factors are believed to be structurally analogous to antibodies, but on a much smaller molecular scale. Both antigen-specific transfer factors and antibodies include antigen-specific sites. In addition, both transfer factors and antibodies include highly conserved regions that interact with receptor sites on their respective effector cells. In transfer factor and antibody molecules, a third, “linker,” region connects the antigen-specific sites and the highly conserved regions.

### The Role of Transfer Factor in the Immune System

Transfer factor is a low molecular weight isolate of lymphocytes. Narrowly, transfer factors may have specificity for single antigens. U.S. Pat. Nos. 5,840,700 and 5,470,835, both of which issued to Kirkpatrick et al. (hereinafter collectively referred to as “the Kirkpatrick Patents”), disclose the isolation of transfer factors that are specific for certain antigens. More broadly, “specific” transfer factors have been generated from cell cultures of monoclonal lymphocytes. Even if these transfer factors are generated against a single pathogen, they have specificity for a variety of antigenic sites of that pathogen. Thus, these transfer factors are said to be “pathogen-specific” rather than antigen-specific. Similarly, transfer factors that are obtained from a host that has been infected with a certain pathogen are pathogen-specific. Although such preparations are often referred to in the art as being “antigen-specific” due to their ability to elicit a secondary immune response when a particular antigen is present, transfer factors having different specificities may also be present. Thus, even the so-called “antigen-specific,” pathogen-specific transfer factor preparations may be specific for a variety of antigens.

Additionally, it is believed that antigen-specific and pathogen-specific transfer factors may cause a host to elicit a delayed-type hypersensitivity immune response to pathogens or antigens for which such transfer factor molecules are not specific. Transfer factor “draws” at least the non-specific T-cells, the T-inducer and T-suppressor cells, to an infecting pathogen or antigenic agent to facilitate a secondary, or delayed-type hypersensitivity, immune response to the infecting pathogen or antigenic agent.

Typically, transfer factor includes an isolate of proteins having molecular weights of less than about 10,000 daltons (D) that have been obtained from immunologically active mammalian sources. It is known that transfer factor, when added either in vitro or in vivo to mammalian immune cell systems, improves or normalizes the response of the recipient mammalian immune system.

The immune systems of newborns have typically not developed, or “matured,” enough to effectively defend the newborn from invading pathogens. Moreover, prior to birth, many mammals are protected from a wide range of pathogens by their mothers. Thus, many newborn mammals cannot immediately elicit a secondary response to a variety of pathogens. Rather, newborn mammals are typically given secondary immunity to pathogens by their mothers. One way in which mothers are known to boost the immune systems of newborns is by providing the newborn with a set of transfer factors. In mammals, transfer factor is provided by a mother to a newborn in colostrum, which is typically replaced by the mother's milk after a day or two. Transfer factor basically transfers the mother's acquired, specific (i.e., delayed-type hypersensitive) immunity to the newborn. This transferred immunity typically conditions the cells of the newborn's immune system to react against pathogens in an antigen-specific manner, as well as in an antigen- or pathogen-nonspecific fashion, until the newborn's immune system is able on its own to defend the newborn from pathogens. Thus, when transfer factor is present, the immune system of the newborn is conditioned to react to pathogens with a hypersensitive response, such as that which occurs with a typical delayed-type hypersensitivity response. Accordingly, transfer factor is said to “jump start” the responsiveness of immune systems to pathogens.

Much of the research involving transfer factor has been conducted in recent years. Currently, it is believed that transfer factor is a protein with a length of about forty-four (44) amino acids. Transfer factor typically has a molecular weight in the range of about 3,000 to about 6,000 Daltons (Da), or about 3 kDa to about 6 kDa, but it may be possible for transfer factor molecules to have molecular weights outside of this range. Transfer factor is also believed to include three functional fractions: an inducer fraction; an immune suppressor fraction; and an antigen-specific fraction. Many in the art believe that transfer factor also includes a nucleoside portion, which could be connected to the protein molecule or separate therefrom, that may enhance the ability of transfer factor to cause a mammalian immune system to elicit a secondary immune response. The nucleoside portion may be part of the inducer or suppressor fractions of transfer factor.

The antigen-specific region of the antigen-specific transfer factors is believed to comprise about eight (8) to about twelve (12) amino acids. A second highly-conserved region of about ten (10) amino acids is thought to be a very high-affinity T-cell receptor binding region. The remaining amino acids may serve to link the two active regions or may have additional, as yet undiscovered properties. The antigen-specific region of a transfer factor molecule, which is analogous to the known antigen-specific structure of antibodies, but on a much smaller molecular weight scale, appears to be hyper-variable and is adapted to recognize a characteristic protein on one or more pathogens. The inducer and immune suppressor fractions are believed to impart transfer factor with its ability to condition the various cells of the immune system so that the cells are more fully responsive to the pathogenic stimuli in their environment.

### Sources of Noncellular Immune System Components

Conventionally, transfer factor has been obtained from the colostrum of milk cows. While milk cows typically produce large amounts of colostrum and, thus, large amounts of transfer factor over a relatively short period of time, milk cows only produce colostrum for about a day or a day-and-a-half every year. Thus, milk cows are neither a constant source of transfer factor nor an efficient source of transfer factor.

Transfer factor has also been obtained from a wide variety of other mammalian sources. For example, in researching transfer factor, mice have been used as a source for transfer factor. Antigens are typically introduced subcutaneously into mice, which are then sacrificed following a delayed-type hypersensitivity reaction to the antigens. Transfer factor is then obtained from spleen cells of the mice.

While different mechanisms are typically used to generate the production of antibodies, the original source for antibodies may also be mammalian. For example, monoclonal antibodies may be obtained by injecting a mouse, a rabbit, or another mammal with an antigen, obtaining antibody-producing cells from the mammal, then fusing the antibody-producing cells with immortalized cells to produce a hybridoma cell line, which will continue to produce the monoclonal antibodies throughout several generations of cells and, thus, for long periods of time.

Antibodies against mammalian pathogens have been obtained from a wide variety of sources, including mice, rabbits, pigs, cows, and other mammals. In addition, the pathogens that cause some human diseases, such as the common cold, are known to originate in birds. As it has become recognized that avian (i.e., bird) immune systems and mammalian immune systems are very similar, some researchers have turned to birds as a source for generating antibodies.

U.S. Pat. No. 6,468,534, issued to Hennen et al. on Oct. 22, 2002 (hereinafter “the '534 Patent”), discloses methods for obtaining transfer factor from the eggs of nonmammalian source animals, including chickens. The method that is described in the '534 Patent includes exposing the nonmammalian source animal to one or more antigenic agents. These antigenic agents have been found to elicit a cell-mediated immune response which includes the production of transfer factor. The transfer factor is present in and may be obtained from the eggs of the source animal. Accordingly, the method of the '534 Patent includes collecting eggs from the nonmammalian source animal.

### Administration of Transfer Factor

While transfer factor from such sources is known to facilitate and enhance a subject's cell-mediated immune response to invasion by pathogens, it has been believed that transfer factor enhances the activity of the so-called “T-natural killer” cells, which produce oxidants. It is well known that, by producing oxidants, T-natural killer cells produce conditions which are not favorable to infecting pathogens and, thereby, “kill” the invading pathogens. Additionally, the high oxidant concentration conditions that are created by T-natural killer cells are also damaging to the cells of the infected subject. Thus, in addition to ridding the subject of pathogen, the cell-mediated immune response of a subject increases oxidative stress in the body of the subject (e.g., by increasing the number of oxidants in the body and, thus, production of antioxidants by the body) and has a somewhat adverse affect on the subject's own cells and tissues. By administering transfer factor to a subject, it has been thought that the cell-mediated immune response would be increased, along with a consequent increase in damage to the treated subject's body.

There are needs for methods and compositions that facilitate the ability of a subject to rid itself of unwanted infections, as well as enhance or sustain, rather than exacerbate, the oxidative balance (i.e., the balance between oxidants and antioxidants) of the subject's body and the ability of the subject's body to heal itself.

### SUMMARY OF THE INVENTION

The present invention includes methods and compositions for focusing the cell-mediated immune response of a subject, such as a mammal (e.g., a livestock, a human, etc.), a bird (e.g., a chicken), or another animal, to an infecting pathogen. The present invention also includes methods and compositions for enhancing or sustaining one or more of a subject's antioxidant profile, detoxification abilities, and general cell and molecular health.

In particular, a method according to the present invention includes administering transfer factor to an infected individual. The transfer factor, which may be derived from a mammalian or nonmammalian (e.g., avian, amphibian, reptilian, etc.) source, may be administered alone or with other suitable therapies, which are effected with known biologically active agents (e.g., antibiotics, antiparasitics, antiviral agents, cytokines, etc.). It has been unexpectedly discovered that by administering transfer factor to an infected subject the subject's oxidant levels do not increase. Instead, even though transfer factor improves the subject's cell-mediated immune response, the oxidant levels are decreased. Thus, transfer factor is believed to focus the cell-mediated immune response of a subject rather than to generally increase the cell-mediated immune response, while maintaining a healthy oxidative balance.

In addition, improvements in the antioxidant profiles of various subjects have been accelerated following administration of transfer factor, relative to the rates of improvement in the antioxidant profiles of subjects who were not treated with transfer factor. It has also been discovered that the abilities of the bodies of subjects that have been treated with transfer factor to self-detoxify is enhanced relative to the abilities of the bodies of untreated subjects to detoxify themselves. As such, the present invention includes a method for improving the antioxidant and detoxification profile of a subject by treating the subject with transfer factor.

The infection-affected cells and tissues of subjects who have been treated with transfer factor also appear to repair themselves more effectively than do the cells and tissues at or near the infection sites of subjects that have not been treated with transfer factor. Accordingly, the present invention includes methods for enhancing the ability of a subject's body to repair its cells by administering transfer factor to the subject.

Likewise, subjects that have been treated with transfer factor and that are recovering from infections evidence greater molecular health than do untreated subjects who are recovering from similar infections. In particular, the overall “health,” as measured by the ratio of reduced forms to oxidized forms, of both proteins and lipids in subjects that are recovering from infections and who have been treated with transfer factor is better than the health of proteins and lipids in subjects who are recovering from similar infections without having been treated with transfer factor. Accordingly, the present invention includes a method for improving the molecular health of a treated subject which includes administering transfer factor to the treated subject. By way of example only and not to limit the scope of the present invention, the invention includes methods for improving the health of a subject's proteins and lipids by administering transfer factor to the subject.

The present invention also includes compositions that are useful for effecting the method of the present invention. In particular, transfer factor and compositions which include transfer factor are within the scope of the present invention. The transfer factor may be derived from any suitable source, such as from the cells of an animal, the colostrum or milk of a mammal, or from eggs.

Other features and advantages of the present invention will become apparent to those of skill in the art through consideration of the ensuing description and the accompanying claims.

### DETAILED DESCRIPTION

Those who understand the role of transfer factor in facilitating cell-mediated immune responses know that transfer factor typically increases the activity of T-cells. It has also been recently shown that transfer factor increases the effectiveness of natural killer cells. Additionally, it is believed that transfer factor enhances the response of cytotoxic T-lymphocytes (CTLs) to infections. It is also well known to those in the art that immune cells, such as neutrophils, produce peroxide and other oxidants in infected regions of the body to “kill” invading pathogens. Thus, it would be expected that by administering transfer factor to a subject, the resulting affect on the subject's cell-mediated immune response would increase the levels of oxidants at or near the site of infection and, thus, result in an increase in the levels of antioxidants produced by the subject's body.

Research has demonstrated otherwise. In particular, it appears that transfer factor may be used to focus the cell-mediated immune response of subjects to invading pathogens. It also appears that administering transfer factor to an subject may enhance and/or increase the efficiency of an subject's various antioxidant systems, permitting the antioxidant systems of the subject to recover more quickly than if transfer factor were not administered. Also, the ability of the subject's body to eliminate toxins appears to be improved by administering transfer factor to the subject. Additionally, it has been discovered that administration of transfer factor to subjects has beneficial affects on the general health of the biomolecules (e.g., proteins, lipids, etc.), cells, and tissues in the treated subject's body.

The following EXAMPLES summarize studies which have been conducted to show these novel and inventive uses for transfer factor.

### EXAMPLE 1

In a first example, the affects of transfer factor on patients with osteomyelitis were evaluated. Osteomyelitis is caused as pyrogenic (i.e., fever-causing) bacteria infect bones. The presence of such an infection typically causes a significant increase in the cell-mediated (i.e., T-cell or leukocyte) immune response at or near the site of infection, which results in an increase in the number of oxidants (e.g., free radicals, peroxides, etc.) at and near the site of the infection. Moreover, when it becomes necessary to remove osteomyelitis by surgery, the trauma that surgery causes results in a heightened cell-mediated immune response which, in turn, leads to even higher levels of oxidants at and near the site of infection. As a consequence of increased levels of oxidants, cellular and bone tissue damage occurs In addition, the concentration of toxins at the location of infected and decaying cells and bone tissue is usually relatively high.

Various characteristics of two groups of infected individuals were evaluated and compared with the characteristics of a sampling of “normal” individuals from the same geographic region. The thirteen (13) individuals in the first group were less sick (i.e., had less extensive infections) than the twenty (20) individuals of the second group. Thus, the individuals of the first group were at a different “healthiness baseline” than the individuals of the second group as the study was initiated.

Administration of transfer factor to each of the individuals of the second group was initiated one week prior to surgery. The treated individuals were each provided with two capsules of TRANSFER FACTOR from 4Life Research, LC, of Sandy, Utah, three times daily, throughout the course of the evaluation.

The individuals of the second group received no such pre-surgery transfer factor treatment.

All of the individuals of both the first group and the second group underwent conventional antibiotic treatment and surgery to remove their infections. Following surgery, the osteomyelitis patients of both the first and second groups received four to six weeks of conventional antibiotic treatment (e.g., gentamycin, ampiox, etc.).

Each of the individuals were evaluated one week before surgery (i.e., at a “baseline” before the individuals in the second group had received transfer factor), one week after surgery, and four weeks following surgery. The ascorbic acid level, thiosulfide antioxidant system (AOS), superoxide dismutase (SOD), glutathioneperoxidase (GPO), catalase, glutathione-s-transferase (G-S-T), malondialdehyde (MDA) level, and protein sulfhydryl (SH) and protein disulfide (SS) groups of each individual were evaluated, as was the cellular membrane integrity as indicated by the erythrocyte stability profiles.

As shown in TABLE 1, the antioxidant abilities of the individuals in the first and second groups were evaluated. In particular, the ascorbate and thiol antioxidant systems of the individuals, respectively referred to in TABLE 1 as “Ascorbate AOS” and “Thiol AOS,” were evaluated. In addition, the levels of various antioxidant enzymes, including SOD, GPO, and catalase, were checked. Levels of G-S-T, an enzyme responsible for removing toxins from the body, were also measured. Protein peroxidation levels were also evaluated.

The data in TABLE 1 represents average levels of each of the characteristics that were measured in both groups of individuals.

TABLE 1
Indices of the body non-specific resistance in osteomyelitis patients taking TF

| | Control group | | | Test group | | |
|---|---|---|---|---|---|---|
| | | 1 Week | 4 Weeks | | 1 Week | 4 Weeks |
| Groups | Before | after | after | Before | after | after |
| Indices | treatment | surgery | surgery | treatment | surgery | surgery |

Low molecular weight antioxidants

| | | | | | | | |
|---|---|---|---|---|---|---|---|
| Ascorbate | Tf | 26.0 ± 4.1 | 13.5* ± 3.5 | 17.0* ± 5.1 | 14.0• ± 4.3 | 18.0• ± 5.2 | 16 ± 4.3 |
| AOS | (Mg/l) | | | | | | |
| | Of | 18.5 ± 5.0 | 10.5* ± 3.1 | 15 ± 3.1 | 12.0 ± 3.0 | 11.2 ± 2.2 | 15 ± 3.3 |
| | (Mg/l) | | | | | | |
| | Rf | 5.1 ± 0.7 | 4.5 ± 0.6 | 2.8* ± 0.9 | 2.0• ± 0.5 | 4.0 ± 0.7 | 3.6 ± 0.8 |
| | (Mg/l) | | | | | | |
| | Rf/Of | 0.24 | 0.50 | 0.21 | 0.17 | 0.28• | 0.34* |
| Thiol | SH | 1.36 ± 0.41 | 1.28 ± 0.35 | 1.24 ± 0.28 | 1.20 ± 0.25 | 1.12 ± 0.18 | 1.38 ± 0.19* |
| AOS | (MM/l) | | | | | | |
| | SS | 0.50 ± 0.06 | 0.52 ± 0.07 | 0.44 ± 0.06 | 0.44 ± 0.05 | 0.44 ± 0.05 | 0.38 ± 0.06 |
| | (MM/l) | | | | | | |
| | SH/SS | 2.5 | 2.4 | 2.8 | 2.7 | 2.4 | 3.6 |

Enzymatic AOS link

| | | | | | | |
|---|---|---|---|---|---|---|
| SOD | 63.0 ± 15.1 | 32.6* ± 9.0 | 59.0 ± 14.3 | 53.0 ± 16.0 | 47.0 ± 19.0 | 34.0* ± 13.1 |
| (activity/g. sec.) | | | | | | |
| Catalase | 1020 ± 220 | 757* ± 186 | 1200 ± 235 | 784 ± 130 | 790 ± 142 | 1022 ± 141 |
| (MM/g. sec.) | | | | | | |
| GPO | 570 ± 90 | 579 ± 95 | 535 ± 105 | 709• ± 120 | 511 ± 111 | 542* ± 123 |
| (MM/g. sec.) | | | | | | |
| G-S-T | 53 ± 9.8 | 45 ± 10.6 | 67 ± 12.5 | 21• ± 11.3 | 47 ± 10.1 | 57.0* ± 10.7 |
| (MM/g. sec.) | | | | | | |
| General protein | 86.0 ± 12.1 | 94.0 ± 14.3 | 82.5 ± 13.8 | 88.0 ± 12.9 | 97 ± 13.0 | 97 ± 14.0 |
| hemolysate | | | | | | |
| (×10−4 g/ml) | | | | | | |

Protein peroxidation

| | | | | | | |
|---|---|---|---|---|---|---|
| SH (MM/l) | 7.3 ± 2.1 | 7.1 ± 2.0 | 7.0 ± 1.9 | 6.72 ± 1.5 | 6.84 ± 1.6 | 7.8• ± 1.5 |
| SS (MM/l) | 3.0 ± 0.5 | 2.9 ± 0.4 | 2.4 ± 0.4 | 2.56 ± 0.45 | 2.7 ± 0.38 | 2.1 ± 0.4 |
| SH/SS | 2.4 | 2.4 | 3.2 | 2.5 | 2.35 | 3.7•* |

•statistically significant differences (p ≦ 0.05) as compared with the control group indices
*statistically significant differences (p ≦ 0.05) as compared with the indices in the group before the treatment

From the data in TABLE 1, several of the affects of transfer factor on an infected individual can be seen.

As one example, the oxidized (Of), reduced (Rf), and total (Tf) ascorbate (i.e., vitamin C) fractions were evaluated. The ratio of the reduced ascorbate fraction to the oxidized ascorbate fraction, or ratio, (Rf/Of) was then determined. The Rf/Of fraction is particularly significant since it provides information about the ability of a subject's body to reduce oxidant levels. More specifically, the reduced form of ascorbate, especially when present in high concentrations, acts as a chemical antioxidant by inactively reacting with oxidants, such as peroxides and free radicals. When oxidants are more likely to react with a chemical antioxidant, such as the reduced form of ascorbate, than proteins, lipids, and other biomolecules, particularly those which are present on or in cell membranes, the incidence of damage to cells and tissues in a subject's body are less likely to be damaged.

In the geographical region in which these tests were conducted, the Rf/Of ratio of a healthy individual will normally be in the range of about 0.6 to about 0.8. Notably, the Rf/Of ratios in the individuals of the second group (0.17) were initially much lower than the initial Rf/Of ratios of the individuals in the first group (0.24), indicating that, prior to transfer factor treatment, surgery, and antibiotic treatment, the individuals in the second group were initially sicker than the individuals in the first group.

Moreover, while the Rf/Of ratio does not appear to have increased for the individuals of the first group, who were not treated with transfer factor (the final average was 0.21), which was not unexpected following surgery, a significant, two-fold, increase in the Rf/Of ratio (to 0.34) was seen in individuals who were treated with transfer factor (i.e., those in the second group). This increase in the Rf/Of ratio of the treated individuals was completely unexpected since transfer factor is known to boost the cell-mediated immune response and, thereby, would have been expected to cause an increased oxidant level and, thus, a decrease in the Rf/Of ratio. These results suggest that transfer factor actually enhances the ascorbate AOS of treated individuals.

When taken in connection with information that suggests that the overall health of the bodies of individuals who have been treated with transfer factor has improved over the same period of time, which is discussed below in reference to TABLE 2, it can be seen that this apparent decrease in oxidant levels is due to a decreased need for a cell-mediated immune response.

Data that was obtained with respect to the thiol AOSs of the individuals who participated in the study likewise shows that individuals who were treated with transfer factor (i.e., individuals in the second group) exhibited an increase the ratio of reduced thiols (SH), such as glutathione and cysteine, to oxidized thiols (SS), whereas no significant change in this ratio was seen in the individuals of the first group. Again, the increase in the reduced forms (SH) of the molecules that participate in the thiol AOS was unexpected, as transfer factor is known to improve an individual's cell-mediated immune response and, thus, would be expected to result in significantly increased oxidant levels.

Like the reduced form of ascorbate, reduced thiols (SH) act as chemical “sponges” that react with oxidants in the body to prevent oxidation of proteins and other biomolecules, including those which are present on and in cell membranes. Accordingly, relatively high SH/SS ratios indicate that the general cellular health of an individual is good.

When taken along with information that indicates that the overall cellular and molecular health of the individual has improved, as discussed in reference to TABLE 2, the increase in the ratio of reduced to oxidized sulfides indicates a decreased need for a cell-mediated immune response.

Additionally, the information that was obtained about the ascorbate and thiol AOSs of the evaluated individuals indicates that the AOSs of those in the second group, who had been treated with transfer factor, more quickly approach “normal” activity than the antioxidant systems of individuals in the first, untreated group.

In addition, TABLE 1 shows SOD and GPO levels that were measured in both the first, untreated, and second, transfer factor-treated groups of individuals at one week prior to surgery, one week following surgery, and four weeks following surgery. SOD and GPO levels appear to have decreased slightly in the first group, while levels of these antioxidant enzymes decreased more significantly in the individuals of the second group, who were treated with transfer factor. As known in the art, the production of antioxidant enzymes by a subject is typically increased as the levels of oxidants in the body of the subject increase. Conversely, as oxidant levels in the body of a subject decrease, high levels of antioxidant enzymes are no longer needed and antioxidant enzyme production decreases. Accordingly, the significant decreases in the SOD and GPO levels of the individuals who were treated with transfer factor (i.e., the second group) indicates that transfer factor improved or enhanced (e.g., toward “normal” levels or better) the efficiency with which the antioxidant systems of these individuals worked to remove oxidants from their bodies.

It is believed that transfer factor may increase the efficiency of a subject's antioxidant systems by one or more of three mechanisms. For example, transfer factor may “lead” natural killer cells to focus more directly on the invading pathogen. As another example, transfer factor may protect the membranes of the cells of an infected subject. Another exemplary mechanism by which transfer factor may increase the efficiency of a subject's antioxidant systems is by actively assisting antioxidants.

At low levels, catalase works as an antioxidant. At higher levels, however, such as those seen in TABLE 1 with respect to individuals who had been treated with transfer factor, catalase is known to detoxify the body.

TABLE 1 also shows that the activity of G-S-T, a detoxification enzyme, increased in both the first and second groups of individuals. The mechanism by which G-S-T detoxifies is well known: it binds toxins to glutathione, a solubilizing agent which carries otherwise insoluble toxins out of the body. While the measured increases in G-S-T activity were significant in both the first group and the second group, G-S-T activity increased to a much greater extent in the individuals of the second group than in the individuals of the first group. As GPO and G-S-T share the same intermediate, glutathione, G-S-T levels typically do not increase until there is a corresponding decrease in the amount of GPO present. Accordingly, the increase in G-S-T levels of an individual who has been treated with transfer factor indicates that GPO production is no longer needed to reduce oxidant levels and, thus, that the focus of the body's repair efforts has shifted from reducing oxidant levels to detoxification, or removal of toxins, xenobiotics, “dead” cells, pathogens, and damaged biomolecules. The significantly larger G-S-T levels in the individuals of the second group, to whom transfer factor was administered, indicates that the bodies of these individuals were more efficiently detoxifying themselves. Also, based on the G-S-T measurements that are provided in TABLE 1, it appears that transfer factor decreases the amount of time it takes the body of a treated subject to switch over to the detoxification process.

In view of these results, the present invention also includes administering transfer factor to a subject to increase the efficiency (e.g., to “normal” levels or better) with which the subject's body detoxifies itself as well as to decrease detoxification time.

Finally, TABLE 1 includes information about the affect of transfer factor on the “health” (i.e., oxidation) of proteins. In particular, TABLE 1 illustrates that the ratio of reduced sulfhydryl groups on proteins to oxidized sulfhydryl groups on proteins increased in both the first, untreated group and in the second, transfer factor-treated group. The increase in this ratio was more significant, however, in the individuals of the second group, to whom transfer factor was administered, than in the individuals of the first group. As such, it appears that transfer factor is at least partially responsible for preventing protein oxidation and, thus, for improving the overall “health” of the proteins of a subject that has been treated therewith.

TABLE 2 shows the stability of the membranes of and, thus, the cellular health of erythrocytes (i.e., red blood cells, or rbc's) of the individuals in both the first group and the second group. Erythrocyte stability is an indicator of cellular stability throughout the body of a tested individual. The stability of erythrocytes was measured by exposing them to free radicals, or oxidants. The erythrocyte resistance test is performed to provide an indication of the overall cellular health of an individual who is suffering from a severe infection, such as osteomyelitis. In the erythrocyte resistance test that TABLE 2 illustrates, five categories of erythrocytes are set forth, including prehemolysis, which includes the percentage of erythrocytes that were lysed, or broken, prior to being exposed to free radicals, or oxidants. The remaining four categories of erythrocyte health are based on their relative stabilities when exposed to free radicals, or oxidants over time.

TABLE 2
Blood erythrocytes resistance (B %) of osteomyelitis patients

| | Control group | | | Test group | | |
|---|---|---|---|---|---|---|
| | | | 4 Weeks | | | 4 Weeks |
| Groups | Before | 1 Week after | after | Before | 1 Week after | after |
| Indices | treatment | surgery | surgery | treatment | surgery | surgery |
| Prehemolysis | 1.9 | 2.5 | 3.3 | 2.6 | 4.5 | 6.2 |
| Low stable | 21 | 48 | 68* | 63• | 58 | 51• |
| Moderately | 58.7 | 44 | 25* | 31• | 23• | 38• |
| stable | | | | | | |
| Higher stable | 5.2 | 4.0 | 3.9• | 5.2 | 4.9 | 7.9• |
| Highly stable | 0.02 | 0 | 0 | 0.02 | 0.02 | 0.07• |

•statistically significant differences (p ≦ 0.05) as compared with the control group indices
*statistically significant differences (p ≦ 0.05) as compared with the indices in the group before the treatment

The information which is provided in TABLE 2 indicates that, as of one week before surgery, the cellular health of the individuals in the first group, who were not to be treated with transfer factor, was better than the cellular health of the individuals in the second group, who were to be treated with transfer factor. In particular, TABLE 2 indicates that about 66% of the erythrocytes of the individuals in the first group were at least moderately stable, while the about 66% of the erythrocytes of the individuals in the second group were of low stability or worse at the same relative point in time. The overall stability of erythrocytes in the individuals of the first group appears to have decreased four weeks following surgery, as would be expected following a traumatic event such as surgery. In contrast, the overall stability of erythrocytes of the individuals in the second group, who had been treated with transfer factor, appears to have increased by four weeks after surgery. Thus, based on the data which is provided in TABLE 2, treatment with transfer factor appears to improve cellular stability and, thus, cellular health.

TABLE 3 provides data on the MDA levels of the individuals of the first and second groups, which provides an indication of the blood plasma lipid peroxidation (LPO), or the rate at which fats in the blood are oxidized.

TABLE 3
Blood Plasma Lipid Peroxidation (LPO) in Osteomyelitis Patients.
LPO (by MDA (nmoles/mole))

| | Before | 1 Week after | 4 Weeks after |
|---|---|---|---|
| | treatment | surgery | surgery |
| Control | 2.90 ± 1.17 | 3.56 ± 0.81 | 3.78 ± 1.21 |
| Test | 3.93 ± 1.93 | 3.31 ± 1.32 | 3.38 ± 1.48 |

The “Before treatment” levels of MDA shown in TABLE 3 indicate that MDA levels were higher in the patients of the second group prior to being treated with transfer factor and, thus, that the fats in the blood of the individuals of the second group were oxidized to a greater extent than were the fats in the blood of the individuals of the first group. Based on this information, it can be seen that, prior to transfer factor administration and surgery, individuals of the second group were sicker than individuals of the first group. Looking at the data that was obtained one week and four weeks after surgery, opposite trends are seen: oxidation of blood fats in the individuals of the first group increased, while oxidation in the blood fats of the individuals of the second group decreased. From these results, it is evident that the fats of the individuals of the first group became more sickly, while the lipid “health” of the individuals of the second group improved.

Transfer factor is believed to be responsible for improving (e.g., to “normal” levels or better) the lipid oxidation levels of a subject and, thus, in improving the overall lipid health of a subject. As such, the present invention includes methods for improving the lipid profiles, or health, of a subject by administering transfer factor to the subject.

### EXAMPLE 2

In a second example, the affects of transfer factor on hepatitis patients, including individuals who had been infected with the hepatitis-B virus (HBV) and individuals who had been infected with the hepatitis-C virus (HCV) were studied. The form of viral hepatitis which is caused by HBV causes about two million deaths every year. About two-hundred million people, or about three percent (3%) of the population of the world, are infected with HCV.

In viral infections, such as viral hepatitis, viruses invade one or more specific types of target cells. In the cases of HBV and HCV, the targeted cells are liver cells, or “hepatocytes.” Upon invading a target cell, viruses typically “take over” at least some of the functionality of the cell, often causing the cell to produce more virus particles, then eventually killing the cell as the virus particles are released therefrom.

In addition, nearby uninfected cells may be indirectly affected by viral infections. This is particularly true in the case of HBV infections, in which most of the damage to the liver is caused by the infected host's own immune system. When cells are damaged by a viral infection or by the host's immune system, the cells release many of their contents, including enzymes, other proteins, nucleic acids, and some of their organelles. As some of the enzymes that are released from a dying or dead cell are typically present only when cell death has occurred, these enzymes may be relied upon a indicators of cell death. Alanine amino transferase (AlAT) and aspartate aminotransferase (AsAT) are two examples of such indicator enzymes. A measure of the amounts of these enzymes in the blood serum of a subject is typically indicative of the level of cell death occurring in that subject.

Indicator enzyme levels were evaluated in three groups of patients who were suffering from acute HBV infections. The first group included fifteen patients under conventional care (aimed at improving bile secretion and liver metabolism) and to whom one capsule of TRANSFER FACTOR had been administered three times daily for fourteen days. One capsule of TRANSFER FACTOR PLUS, also available from 4Life Research, was administered to the fourteen patients of the second group three times daily for fourteen days. None of the patients of the first or second groups received interferon (a cytokine) treatment. The third group included fifteen patients who received conventional acute HBV infection care, along with interferon treatment. Each group included a similar “cross-section” (i.e., gender, age, etc.) of patients.

Levels of AlAT and AsAT in the serum of each of these patients were measured during the course of their treatment with TRANSFER FACTOR, interferon, and TRANSFER FACTOR PLUS. On average, the patients of the first group exhibited elevated levels of one or both of AlAT and AsAT for 9.2±0.05 days and the levels of AlAT and/or AsAT were above normal in patients of the second group for 10.1±0.91 days, while AlAT and/or AsAT levels in the serum of the patients of the third group, who had been treated with interferon, remained elevated for an average of 12.2±0.80 days. These results indicate that the transfer factor in both TRANSFER FACTOR and TRANSFER FACTOR PLUS resulted in remission of the symptoms of acute HBV patients in a significantly shorter period of time than interferon treatment caused remission in similar patients.

These results further indicate that transfer factor improves cell stability, as well as the general cellular health of a treated subject.

Moreover, treatment regimen that includes transfer factor appears to have been better tolerated by patients than interferon therapy. In particular, all of the patients who had been treated with transfer factor reported a significant improvement of their general state, including lack of excessive fatigue and the absence of discomfort at the locations of their livers.

### EXAMPLE 3

The affects of transfer factor on patients suffering from opisthorchiasis were evaluated in a third example. Opisthorchiasis, which occurs in Eastern and Central Europe, Siberia, and parts of Asia, is caused in mammals, including humans, dogs, and cats, by one of two types of flukes in the infectious metacercaria stage. Mammals typically contract opisthorchiasis by eating raw or undercooked fish.

An immune imbalance is known to be typical in subjects that are chronically ill with opisthorchiasis.

Forty-five (45) individuals with chronic opisthorchiasis were split into two groups: a first group including twenty-five (25) individuals and a second group including twenty (20) individuals. The individuals of both groups received conventional praziquantel treatment, an anti-parasitic, or antihelminthic, drug which is used in the treatment of opisthorchiasis and is available under the trade name BILTHRICIDE™ from Bayer AG of Leverkusen, Germany. In addition to praziquantel, two capsules of TRANSFER FACTOR PLUS were administered to the individuals of the first group following praziquantel treatment, three times daily for seven days. The individuals of the second group were only treated with praziquantel.

Levels of various cytokines, including γ-interferon (IFN-γ), antibodies, and immune complexes were determined, by known processes, for each the individuals prior to therapy and two weeks following TRANSFER FACTOR PLUS therapy in the individuals of the second group was discontinued. The following TABLE 4 lists the collective measures of IFN-γ in both groups, as determined by use of the ProCon IFN-γ assay available from Protein Contour of St. Petersburg, Russia, and photometrically measured at a wavelength of 492 nm. TABLE 4 also includes a collective measure of the IFN-γ levels of fifteen (15) “normal” blood donors.

TABLE 4
IFN-γ Levels in Chronic Opisthorchiasis Patients

| | First Group | | Second Group | |
|---|---|---|---|---|
| | Before | Two weeks | Before | Two weeks |
| Donors | treatment | after treatment | treatment | after treatment |
| 46.2 ± 6.2 | 43.4 ± 3.1 | 96.4 ± 6.1 | 42.9 ± 6.6 | 51.4 ± 6.3 |
| | p > 0.05 | p > 0.05 | p < 0.05 | p > 0.05 |
| | | p¹ > 0.05 | | p¹ < 0.05 |
| | | | | p² < 0.05 |

p - statistically significant differences versus blood donors
p¹ - statistically significant differences prior to and following treatment
p² - statistically significant differences between groups

These data indicate that, when combined with praziquantel therapy, treatment with TRANSFER FACTOR PLUS resulted in a significant increase in levels of IFN-γ in the individuals of the first group. As is well-known in the art, IFN-γ attracts macrophages, activating them to become more efficient at phagocytosing and destroying invading microorganisms. Stated another way, IFN-γ helps focus the immune system of a treated subject, reducing collateral damage (e.g., in the form increased levels of oxidation or otherwise) that might otherwise be caused by the subject's nonspecific immune response.

### EXAMPLE 4

Similar results were seen in a fourth study, in which the affects of transfer factor on urogenital chlamydiosis patients were determined.

Among other cytokine levels, levels of IFN-γ were determined for three groups, each including fifteen (15) individuals, and compared with IFN-γ levels of the aforementioned group of fifteen (15) “normal” blood donors. The individuals of a first group were treated with 500 mg of claritomycin twice daily for ten (10) to fourteen (14) days, 100 mg of doxycyclin once daily for ten (10) days, and 200 mg of ofloxacin twice daily for ten (10) days, with the drugs having been administered in succession. The individuals of the second group received 500 mg of claritomycin twice daily for ten (10) to fourteen (14) days and one capsule of TRANSFER FACTOR PLUS three times each day for ten (10) days, with treatment with the claritomycin and TRANSFER FACTOR PLUS beginning on the same day. In the third group, each individual was treated with 500 mg of claritomycin twice daily for ten (10) to fourteen (14) days and one capsule of TRANSFER FACTOR thrice daily for ten (10) days, with administration of the claritomycin and TRANSFER FACTOR PLUS having begun on the same day.

Known processes were used to determine IFN-γ levels in the individuals of each of the three groups before the treatment regimen started and following completion of the treatment regimen. The following TABLE 5 lists the collective measures of IFN-γ in all three groups, as determined by use of the ProCon IFN-γ assay available from Protein Contour and photometrically measured at a wavelength of 492 nm. TABLE 5 also includes a collective measure of the IFN-γ levels of fifteen (15) “normal” blood donors.

TABLE 5
IFN-γ Levels in Chlamydia Patients

| | Patients | | | |
|---|---|---|---|---|
| | All three | | | |
| | Groups | First Group | Second Group | Third Group |
| | Before | After | After | After |
| Donors | treatment | treatment | treatment | treatment |
| 46.2 ± 6.2 | 29.4 ± 3.1 | 31.4 ± 6.1 | 102.9 ± 6.6 | 98.4 ± 6.3 |
| | p < 0.05 | p < 0.05 | p < 0.05 | p < 0.05 |
| | | p¹ < 0.05 | | p¹ < 0.05 |
| | | | | p² < 0.05 |

p - statistically significant differences versus blood donors
p¹ - statistically significant differences prior to and following treatment
p² - statistically significant differences between groups following treatment

Similar to the data in EXAMPLE 3, the data of TABLE 5 indicate that, when combined with claritromycin therapy, treatment with transfer factor (in the form of both TRANSFER FACTOR PLUS and TRANSFER FACTOR) resulted in a significant increase in levels of IFN-γ in the transfer factor-treated individuals. Again, it is well-known in the art that IFN-γ is at least partially responsible for focusing the immune system of a treated subject and reducing collateral damage (e.g., in the form increased levels of oxidation or otherwise) that might otherwise be caused by the subject's nonspecific immune response.

Although the foregoing description contains many specifics, these should not be construed as limiting the scope of the present invention, but merely as providing illustrations of some of the presently preferred embodiments. Similarly, other embodiments of the invention may be devised which do not depart from the spirit or scope of the present invention. Features from different embodiments may be employed in combination. The scope of the invention is, therefore, indicated and limited only by the appended claims and their legal equivalents, rather than by the foregoing description. All additions, deletions and modifications to the invention as disclosed herein which fall within the meaning and scope of the claims are to be embraced thereby.

1. A system for restoring an oxidative balance of a body of a subject, comprising at least one biologically active agent and a composition including transfer factor, at least the transfer factor included in an amount tailored to restore the oxidative balance of the body of the subject.

2. The system of claim 1, wherein the transfer factor is specific for a pathogen with which the subject has been infected.

3. The system of claim 1, wherein the composition is substantially free of transfer factor specific for a pathogen with which the subject has been infected.

4. The system of claim 1, wherein the at least one biologically active agent includes at least one of an antibiotic agent, an antiparasitic agent, an antiviral agent, and a cytokine.

5. The system of claim 1, wherein the transfer factor comprises at least one of mammalian transfer factor and avian transfer factor.

6. A system for enhancing an efficiency of at least one enzymatic oxidant of a subject, comprising at least one biologically active agent and a composition including transfer factor, at least the transfer factor included in an amount tailored to enhance the efficiency of the at least one enzymatic oxidant.

7. The system of claim 6, wherein the transfer factor is specific for a pathogen with which the subject has been infected.

8. The system of claim 6, wherein the composition is substantially free of transfer factor specific for a pathogen with which the subject has been infected.

9. The system of claim 6, wherein the at least one biologically active agent includes at least one of an antibiotic agent, an antiparasitic agent, an antiviral agent, and a cytokine.

10. The system of claim 6, wherein the transfer factor comprises at least one of mammalian transfer factor and avian transfer factor.

11. A system for increasing an efficiency with which detoxification proteins of a subject remove toxins from the subject, comprising at least one biologically active agent and a composition including transfer factor, at least the transfer factor included in an amount tailored to increase the efficiency with which detoxification proteins remove toxins.

12. The system of claim 11, wherein the transfer factor is specific for a pathogen with which the subject has been infected.

13. The system of claim 11, wherein the composition is substantially free of transfer factor specific for a pathogen with which the subject has been infected.

14. The system of claim 11, wherein the at least one biologically active agent includes at least one of an antibiotic agent, an antiparasitic agent, an antiviral agent, and a cytokine.

15. The system of claim 11, wherein the transfer factor comprises at least one of mammalian transfer factor and avian transfer factor.

16. A system for improving cellular stability in the body of a subject, comprising at least one biologically active agent and a composition including transfer factor, at least the transfer factor included in an amount tailored to improve the stability of cells in the body of the subject.

17. The system of claim 16, wherein the transfer factor is specific for a pathogen with which the subject has been infected.

18. The system of claim 16, wherein the composition is substantially free of transfer factor specific for a pathogen with which the subject has been infected.

19. The system of claim 16, wherein the at least one biologically active agent includes at least one of an antibiotic agent, an antiparasitic agent, an antiviral agent, and a cytokine.

20. The system of claim 16, wherein the transfer factor comprises at least one of mammalian transfer factor and avian transfer factor.

21. A system for improving molecular health of a subject, comprising at least one biologically active agent and a composition including transfer factor, at least the transfer factor included in an amount tailored improve the molecular health of the subject.

22. The system of claim 21, wherein the transfer factor is specific for a pathogen with which the subject has been infected.

23. The system of claim 21, wherein the composition is substantially free of transfer factor specific for a pathogen with which the subject has been infected.

24. The system of claim 21, wherein the at least one biologically active agent includes at least one of an antibiotic agent, an antiparasitic agent, an antiviral agent, and a cytokine.

25. The system of claim 21, wherein the transfer factor comprises at least one of mammalian transfer factor and avian transfer factor.

26. A method increasing a number of chemical oxidants in a body of a subject, comprising administering to the subject a composition comprising a quantity of transfer factor tailored to increase the number of chemical oxidants in the body of the subject.

27. The method of claim 26, wherein said increasing includes increasing a concentration of a reduced form of ascorbate in the subject.

28. The method of claim 26, wherein said increasing includes increasing a concentration of reduced thiols in the subject.

29. The method of claim 26, wherein said administering comprises administering to the subject a composition comprising transfer factor which is specific for a pathogen with which the subject has been infected.

30. The method of claim 26, wherein said administering comprises administering to the subject a composition which is substantially free of transfer factor specific for a pathogen with which the subject has been infected.

31. A method for enhancing an efficiency of at least one enzymatic antioxidant in a body of a subject, comprising administering to the subject a composition comprising a quantity of transfer factor tailored to enhance the efficiency of the at least one enzymatic antioxidant in the body of the subject.

32. The method of claim 31, wherein said enhancing comprises reducing a concentration of said at least one said enzymatic antioxidant in the subject.

33. The method of claim 31, wherein said enhancing comprises enhancing activity of at least one of a superoxide dismutase and a glutathione peroxidase.

34. The method of claim 31, wherein said administering comprises administering to the subject a composition comprising transfer factor which is specific for a pathogen with which the subject has been infected.

35. The method of claim 31, wherein said administering comprises administering to the subject a composition which is substantially free of transfer factor specific for a pathogen with which the subject has been infected.

36. A method for increasing an efficiency with which detoxification proteins of a body of a subject remove toxins from the body of the subject, comprising administering to the subject a composition comprising a quantity of transfer factor tailored to increase the efficiency with which detoxification proteins remove toxins.

37. The method of claim 36, wherein said administering comprises increasing a concentration of said at least one detoxification protein in the subject.

38. The method of claim 36, wherein said administering comprises increasing a concentration of at least one of a catalase and a glutathione S-transferase in the subject.

39. The method of claim 36, wherein said administering comprises administering to the subject a composition comprising transfer factor which is specific for a pathogen with which the subject has been infected.

40. The method of claim 36, wherein said administering comprises administering to the subject a composition which is substantially free of transfer factor specific for a pathogen with which the subject has been infected.

41. A method for improving cellular stability of a body of a subject, comprising administering to the subject a composition comprising a quantity of transfer factor tailored to improve the stability of cells of the body of the subject.

42. The method of claim 41, wherein said administering comprises decreasing a number of red blood cells that are lysed when exposed to a substantially fixed concentration of at least one antioxidant.

43. The method of claim 41, wherein said administering comprises decreasing a concentration the subject of at least one protein indicator of cell lysis.

44. The method of claim 41, wherein said decreasing comprises decreasing a concentration of at least one of alanine amino transferase and aspartate amino transferase in the subject.

45. The method of claim 41, wherein said administering comprises administering to the subject a composition comprising transfer factor which is specific for a pathogen with which the subject has been infected.

46. The method of claim 41, wherein said administering comprises administering to the subject a composition which is substantially free of transfer factor specific for a pathogen with which the subject has been infected.

47. A method for improving molecular health in a body of a subject, comprising administering to the subject a composition comprising a quantity of transfer factor tailored to improve the molecular health.

48. The method of claim 47, wherein said administering comprises increasing a ratio of a reduced form of protein thiol groups to an oxidized form of protein thiol groups.

49. The method of claim 47, wherein said administering comprise decreasing a measure of oxidized lipids in blood of the subject.

50. The method of claim 47, wherein said administering comprises administering to the subject a composition comprising transfer factor which is specific for a pathogen with which the subject has been infected.

51. The method of claim 47, wherein said administering comprises administering to the subject a composition which is substantially free of transfer factor specific for a pathogen with which the subject has been infected.
